(19) United States
(12) Patent Application Publication
(10) Pub. No.: US 2004/0037896 A1
(43) Pub. Date: Feb. 26, 2004

(54) Therapeutic treatment

(76) Inventor: Pieter Theo Ernst, Northcliff (ZA)

Correspondence Address: BURNS DOANE SWECKER & MATHIS L L P, POST OFFICE BOX 1404, ALEXANDRIA, VA, 22313-1404, US

(21) Appl. No.: 10/433,944

(22) PCT Filed: Dec. 7, 2001
(86) PCT No.: PCT/IB01/02328

(30) Foreign Application Priority Data
Dec. 8, 2000 (ZA) 2000/7313
Feb. 21, 2001 (ZA) 2001/1449

(51) Int. Cl. A61K 33/00; A61K 31/727; A61K 31/714; A61K 31/519

(52) U.S. Cl. 424/717; 514/52; 514/304; 514/56; 514/276; 514/250; 514/356

(57) ABSTRACT

A lignocaine or a salt, ester or metabolite thereof for use in initiating the natural capacity of regeneration of collateral blood vessels.

### BACKGROUND OF THE INVENTION

This invention relates to therapeutic treatment.

EDTA chelation therapy is a well known treatment which involves administering an EDTA solution intravenously to a patient. EDTA chelation therapy has been shown to be useful in the treatment of chronic degenerative diseases and as a therapy before by-pass surgery or angioplasty.

Lignocaine, particularly in the form of the hydrochloride salt, is a known local anaesthetic and anti-antiarrhythmic agent. Lignocaine is also known as lidocaine—The Merck Index, Twelfth Edition, 1996.

### SUMMARY OF THE INVENTION

According to a first aspect of the invention, there is provided lignocaine or a salt, ester, or metabolite thereof, for use in the manufacture of a medicament for use in initiating the natural capacity of regeneration of collateral blood vessels. A collateral blood vessel is a blood vessel which develops along an obstructed blood vessel.

According to a second aspect of the invention, there is provided a method of treating a subject to initiate the natural capacity of regeneration of collateral blood vessels which includes the step of administering to the subject lignocaine or a salt, ester, or metabolite thereof.

According to a further aspect of the invention, there is provided a pharmaceutical composition, particularly for use in the initiation of the natural capacity of regeneration of collateral blood vessels, comprising lignocaine or a salt, ester, or metabolite thereof, a combination of vitamins suitable for cell repair, and a magnesium salt such as magnesium sulphate.

The composition which forms an aspect of the invention has an aqueous carrier and preferably has a pH in the range 6:8, typically about 7. The composition may also contain one or more of ascorbic acid, a bicarbonate and heparin, preferably as the sodium salt. The combination of vitamins is preferably a vitamin B complex comprising thiamine HCl, riboflavin and nicotinamide, and vitamin B12.

The lignocaine or a salt, ester or metabolite thereof, and the composition of the invention is preferably administered to a subject or patient intravenously or intra-arterially. When the compound or composition is administered intravenously, the administration preferably takes place over a period of time, typically a period of one and a half hours or greater. When the compound or composition is administered intra-arterially, the administration is over a much shorter time, for example, less than one minute. The administration may be a combination of intra-arterial and intravenous administrations.

The metabolite of lignocaine will typically be diethylaminoethanol.

### DESCRIPTION OF EMBODIMENTS

An embodiment of an aqueous composition suitable for intravenous or intra-arterial administration to a patient has the components set out in Table 1.

TABLE 1

| | | |
|---|---|---|
| 1 | Ascorbic acid injection 500 mg/5 ml ampoule | 10 ml |
| 2 | Lignocaine HCL anhydrous 20 mg/ml 2% | 15 ml |
| 3 | Magnesium sulphate 50% 1 g/2 ml | 2 ml |
| 4 | Sodium bicarbonate injection 8.5% m/v | 10 ml |
| 5 | Heparin sodium injection 1000 i.u./ml | 1 ml |
| 6 | Vitamin B complex injection | 1.5 ml |
| | Thiamine HCL 10 mg per 1 ml | |
| | Riboflavin 2 mg per 1 ml | |
| | Nicotinamide 100 mg per 1 ml | |
| 7 | Vitamin B 12 injection 1000 μg/ml | 1.5 ml |

pH = 7.35

The composition described above is useful, in particular, for the initiation of the natural capacity of regeneration of collateral blood vessels in a patient. The composition may be administered intravenously or intra-arterially, or a combination of intravenous and intra-arterial administration. When the administration is intravenous, the composition set out in Table 1 will typically be added to a volume of water, e.g. 200 ml, and administered in this form to a patient. The period of administration will be at least one and a half hours. When the composition is administered intra-arterially, the dose is one half that set out in Table 1 administered over a short period of time, e.g. less than one minute.

The composition and compounds of the invention are effective in the treatment of disease that results from poor circulation due to calcified artherosclerotic vessels. Common causes of this disease include hypercholesterolemia, diabetes mellitus, smoking and hypertension. Less common arterial diseases include large and small vasculitis, thromboangiitis obliterans (Buerger's disease).

Further Raynaud's phenomenon/disease and peripheral neuropathy originating from bad circulation are causes of ulceration and severe pain.

Patients who have been successfully treated with the composition of the invention suffered from:

Coronary arterial disease (angina pectoris)

Intermittent claudiocation

Arterial leg ulcers (gangrene)

Cerebral arterial occlusion

Buerger's disease hand and feet

Necrosis/ulcerations of toes, feet and fingers

Venous (varicose vein) ulcers

The following enhancing effects have been noticed in patients receiving the treatment:

An increase of hair growth, even in bald patents.

Increase in the speed of nail growth in hand and feet.

Restoration of erectile dysfunction.

Improved vision and reading capacity especially in the elderly.

Cessation and slightly improving macula degeneration.

Reduce memory loss extensively.

Reduction of the amount and severity of varicose veins.

More particularly, a large number of patients, i.e. over 160, suffering from diseases that result from poor circulation have been treated successfully with the composition described in Table 1. Examples of the treatments are:

1. A patient, 30 years old, had severe gangrene of the right foot, caused by a total occlusion of arteries in the right leg. The patient's history revealed nefrotic syndrome and colitis ulcerosa. The patient also had a cholesterol level of 18.4 mmol/l.

The patient was treated three times weekly by way of intra-arterial administration with an aqueous composition, a dose of one half that set out in Table 1. The patient was further treated once weekly with the composition set out in Table 1, diluted with 200 ml of water, by way of intravenous administration over a period of about one and a half hours for each administration. The improvement in the condition of the patient's right foot was dramatic and after ten months treatment in this manner the leg had healed completely. No further occlusion of the patient's arteries in his right leg have been observed. Further, the cholesterol level of the patient was found to have dropped to 10.4 mmol/l after three months of treatment without the use of any anti-lipid medication. After the leg had healed and the treatment stopped, the cholesterol level of the patient was found to increase significantly. Re-introduction of the intravenous treatment as described above for a period of six months reduced the cholesterol level of the patient to 4 mmol/l.

2. A patient, 78 years old, suffered from severe occlusive arterial disease and small vessel disease. This caused the development of a leg ulcer. The patient was treated with an aqueous composition, one half that set out in Table 1, intra-arterially. Ten separate such administrations of the composition equally spaced over a period of six weeks resulted in the ulcer closing. No further treatment of the patient was necessary.

3. A patient, 42 years old, suffered from occlusive arterial disease. The patient had been advised to have her legs amputated. The patient was treated intra-arterially and intravenously in the manner described for patient (1). Four months after the treatment, the circulation successfully returned to the legs of the patient allowing the patient to lead a normal life.

4. A patient, 54 years old, suffered from continuous chest pain during any effort exerted. The patient had been advised that coronary surgery was probably required. The patient was subjected to forty separate intravenous administrations of the composition set out in Table 1, the composition being diluted with 200 ml of water. Each intravenous administration took place over a period of about one and a half hours. The chest pains have now disappeared and the patient is able to participate in exercises requiring effort without suffering chest pains.

1. Lignocaine or a salt, ester or metabolite thereof for use in the manufacture of a medicament for use in initiating the natural capacity of regeneration of collateral blood vessels.

2. A metabolite of claim 1 which is diethylaminoethanol.

3. A method of treating a subject to initiate the natural capacity of regeneration of collateral blood vessels includes the step of administering lignocaine or a salt, ester or metabolite thereof to the subject.

4. A method according to claim 3 wherein the lignocaine or a salt, ester or metabolite thereof is provided in the form of a composition which includes a combination of vitamins suitable for cell repair and a magnesium salt.

5. A method according to claim 4 wherein the composition has an aqueous carrier and a pH in the range 6 to 8.

6. A method according to claim 5 wherein the composition has a pH of about 7.

7. A method according to any one of claims 4 to 6 which also contains one or more of ascorbic acid, a bicarbonate and heparin.

8. A method according to any one of claims 4 to 7 wherein the combination of vitamins comprises a viatmin B complex comprising thiamine HCl, riboflavin and nicotinamide, and vitamin B12.

9. A method according to any one of claims 3 to 8 which is administered to the subject intravenously.

10. A method according to claim 9 wherein the intravenous administration is carried out over a period of at least one and a half hours.

11. A method according to any one of claims 3 to 8 which is administered to the patient intra-arterially.

12. A pharmaceutical composition comprising lignocaine or a salt, ester, or metabolite thereof, a combination of vitamins suitable for cell repair, and a magnesium salt.

13. A composition according to claim 13 wherein the magnesium salt is magnesium sulphate.

14. A composition according to claim 12 or claim 13 which has an aqueous carrier and a pH in the range 6 to 8.

15. A composition according to claim 14 which has a pH of about 7.

16. A composition according to any one of claims 12 to 15 wherein the combination of vitamins is a vitamin B complex comprising thiamine HCl, riboflavin and nicotinamide, and vitamin B12.

17. A composition according to any one of claims 12 to 16 which further contains one or more of ascorbic acid, a bicarbonate and herparin.

18. A method according to claim 3 substantially as hereinbefore described.

19. A composition according to claim 12 substantially as herein described.
